(19) United States
(12) Patent Application Publication
(10) Pub. No.: US 2024/0325631 A1
(43) Pub. Date: Oct. 3, 2024

(54) CANNULA ASSEMBLY FOR A WEARABLE DRUG DELIVERY DEVICE

(71) Applicant: TecMed AG, Burgdorf (CH)

(72) Inventors: Ursina Streit, Kirchberg (CH); Jan Baumert, Grünen (CH); Michael Hanimann, Bern (CH); Roland Margot, Worb (CH); Fabian Steiner, Burgdorf (CH); Eduard Kobel, Trachselwald (CH)

(21) Appl. No.: 18/615,390

(22) Filed: Mar. 25, 2024

(30) Foreign Application Priority Data
Mar. 30, 2023 (EP) 23165704.0

(51) Int. Cl. A61M 5/142

(52) U.S. Cl. CPC A61M 5/14248; A61M 2207/10

(57) ABSTRACT

A cannula assembly includes a soft cannula, a rigid cannula, and a sealing sleeve. A soft cannula lumen surrounds at least a portion of the rigid cannula and provides a sliding connection. The sealing sleeve radially surrounds at least a portion of the soft cannula. A flange of the soft cannula has an extended wall thickness and is arranged at or near a proximal end of the soft cannula. An inner surface of the sealing sleeve engages an outer surface of the soft cannula and provides a sealing pressure radially between the rigid cannula and the soft cannula, defining a sealing area on an outer surface of the rigid cannula. A drug delivery device with the cannula assembly and an automatic insertion mechanism uses the sealing sleeve to ensure a fluid-tight, slidable connection between the soft cannula and the rigid cannula with improved sealing pressure, while allowing damage-free, cost-effective manufacturing.

### CROSS REFERENCE TO RELATED APPLICATIONS

This application claims priority to European Patent Application No. 23165704.0 filed on Mar. 30, 2023, which is incorporated by reference herein, in its entirety and for all purposes.

### TECHNICAL FIELD

Cannula assemblies for use in a drug delivery device, in particular to infusion pumps or injectors with a cannula insertion mechanism are provided, and include a fluid tight connection between a rigid cannula and a soft cannula.

### BACKGROUND

A variety of diseases exist that require regular treatment by subcutaneous administration of a medicament, and a number of delivery devices have been developed to support a patient in accurately and controllably delivering an amount of drug in a self-administration process. Drug delivery devices include injection devices that are removed from the site of application after each medication event or drug delivery process, as well as infusion devices with a cannula or needle that remains in the skin of the patient for a prolonged period of time. By way of example, diabetes may be treated by administration of insulin by the patients themselves with the help of multi-variable-dose insulin injection pens or infusion pumps. Alternatively, patch injectors, wearable injectors or wearable pumps are patched or adhered to the skin of the patient.

Common to all devices for subcutaneous drug delivery is a reservoir to store the fluid medicament, and a fluid path to bring the drug out of the device and into the subcutaneous tissue of a patient. Many of such devices are designed as patch pumps or patch injectors for attachment to the user's body, where they remain for the duration of injection or infusion as a wearable pump without the need of actively holding the device in place. To further assist the user, patch devices typically include a cannula insertion and retraction mechanism, which allows applying the patch to the skin and removing the device without manual handling of the cannula. The patch device will automatically insert the cannula out of the housing into the body of a patient for delivery of the drug, and also retract the cannula into the housing after an injection or infusion cycle has been terminated. For users' comfort, wearable pumps often use a flexible or soft cannula for drug delivery, which helps users avoiding pain when moving around with a patch pump attached to, and a cannula inserted into the body. A flexible or soft cannula, however, is too soft to pierce the tissue of the body and hence cannot be pushed out of a pump housing and into the tissue of a patient. An extra rigid insertion needle may be needed to support the soft cannula for bringing the soft cannula into delivery position. In a typical design, the rigid cannula is connected to the drug reservoir and provides a fluid connection at the proximal end. The soft cannula is slidingly mounted onto the rigid cannula, with the rigid cannula arranged inside the soft cannula and protruding at the distal end for insertion. After inserting the soft cannula, the rigid insertion needle is retracted into the housing of the patch device, leaving the soft cannula out for drug delivery.

U.S. Pat. No. 6,960,192 B1 describes such a patch pump with a cannula assembly consisting of a soft cannula and a rigid cannula, where the rigid cannula is arranged inside the soft cannula for insertion, and to slidably move axially back inside the soft cannula for drug delivery. No indication is disclosed of how to provide the sealing function between the soft cannula and the rigid cannula.

EP 3928814 A1 gives more details about the design of a patch pump with an insertion mechanism for a soft cannula by using a rigid cannula. Each of the cannulas are attached to a cannula holder movably mounted relative to the pump housing. Before use, the rigid cannula is slidingly arranged inside the soft cannula, with the tip protruding on the distal end, ready to pierce the skin of a patient. To bring the cannula assembly into a position for drug delivery, an insertion spring is triggered to move both cannula holders and hence both cannulas forward into the body of the patient. Once the soft cannula is inserted, a retraction spring moves the rigid cannula holder and also the rigid cannula back into the housing leaving the soft cannula in the tissue. The medicament can now be transferred from the reservoir through the rigid cannula and the soft cannula into the body of the patient. In this document, the sealing between the soft cannula and the rigid cannula is described with an area of extended wall thickness at the proximal end of the soft cannula, and with a flange to provide a proper grip for the soft cannula when it is moved axially, or when an axial force is applied to the soft cannula, for example when removing the patch pump from the infusion site. While this arrangement may be adequate for a lower range of fluid pressure, it is a weak point when it comes to reliability of drug delivery.

For reliable drug delivery, the entire fluid path from the reservoir to the distal end of the cannula needs to be fluid-tight. As also further explained in EP 3928814 A1, monitoring of drug delivery, in particular detection of occlusion, heavily depends on the quality of sealing between the soft cannula and the rigid cannula. To achieve a better sealing is technically linked to applying more sealing pressure, which also makes manufacturing of the needle assembly significantly more difficult because the rigid cannula would possibly damage the inner surface of the soft cannula in the process. A cannula assembly with a weak scaling will typically be easier to manufacture, but will perform worse during drug delivery. Hence, there is clearly a need for a cannula assembly for use in a portable (e.g., mobile) or wearable drug delivery device which provides a slidable and reliably fluid-tight sealing between the soft cannula and the rigid cannula, while still allowing easy and damage-free manufacturing.

In U.S. Pat. No. 6,960,192 a patch pump with a cannula assembly is disclosed, where the flexible cannula has a sealing portion through which the rigid cannula extends. Just as in EP 3928814 A1, no indication is given of how to technically achieve an optimal sealing function in said sealing portion of the soft cannula.

A common approach to solve manufacturing problems including sealing between different components is to bring components together in an uncompressed or lose state, where assembly of the components can reliably be conducted. A manufacture step is then added to bring components in a compressed state, where pressure between components provide a fluid-tight connection where necessary. A press-fit connection of a plug in an opening of another component is a very basic example of such a sealing. However, a press-fit connection has very high friction between components and is not suitable for sliding connection of moving components. Alternatively, a thermic shrinkable soft tube with a large diameter may be pulled over a rigid tube or cannula with a small diameter. Heat is applied to shrink the soft tube from a large diameter to a small diameter until it reaches the surface of the rigid tube. This manufacturing process is very easy and can be done without risk of harming the soft tube. However, for medical devices, in particular for components in contact with the drug, materials need to be bio-compatible, restricting the choice of materials. Furthermore, the elasticity of soft tubes is difficult to control with shrinkable tubes, making them less suitable for providing a slidable sealing.

Another common approach to sealingly connect a soft tube to a rigid tube is to add an outer sleeve to the cannula assembly. The outer sleeve may be constructed from a metal or other material which is plastically deformable by mechanical means. When manufacturing the cannula assembly, both soft and rigid cannula would be introduced into the sleeve, and the sleeve would be mechanically deformed by crimping to apply sealing pressure to the outside of the soft cannula. Again, a drawback is introduced by this method, as the deformation of the sleeve may typically not be uniform around the periphery of the soft cannula, and consequently the sealing pressure inside the soft cannula will vary, resulting in a potentially unreliable sealing. Such an outer sleeve, when used to assemble a soft cannula without a flange, may further not be suitable for sealing of sliding components, as the sleeve may slip off the soft cannula during insertion or retraction of the needle assembly. With a thin wall, the soft cannula may also be prone to damage and consequently to leaking.

### SUMMARY

It is an objective of the invention to provide a wearable drug delivery device with a cannula insertion mechanism and with an improved cannula assembly, which ensures both damage-free and cost-effective manufacturing, and a reliable slidable sealing between a soft cannula and a rigid cannula.

The drug delivery device may be a tubeless patch infusion pump attachable to the skin of a patient by means of an adhesive layer, for subcutaneous delivery of insulin over a period of more than 48 hours through a cannula integrated into the pump. Alternatively, the delivery device is not a tubeless patch infusion pump attachable to the skin of a patient by means of an adhesive layer, for subcutaneous delivery of insulin through a cannula integrated into the pump over a period of more than 48 hours. For instance, the alternative delivery device may be a patch injector attachable to the skin of a patient by means of an adhesive layer, for subcutaneous delivery of a drug other than insulin over a period of less than 48 hours. The alternative delivery device may be a wearable insulin pump or a handheld injection pen devoid of an adhesive layer for attaching to the skin of a patient.

The objective is achieved by introducing a cannula assembly for use in a portable or wearable drug delivery device, comprising a soft cannula, a rigid cannula, and a sealing sleeve, where the soft cannula comprises a wall forming a soft cannula lumen configured to surround or envelope at least a portion of the rigid cannula and to provide a sliding connection between the rigid and soft cannula. The sealing sleeve is configured to radially surround or envelope at least a portion of the soft cannula. The soft cannula includes a soft cannula flange constructed as a portion with an extended wall thickness at, or near the proximal end of the soft cannula. An inner surface of the sealing sleeve may at least partially engage the outer surface of the soft cannula and may provide a radial sealing pressure between the rigid cannula and the soft cannula, thereby defining a sealing area on the outer surface of the rigid cannula. Said sealing area may be defined as the specific part of the outer surface of the rigid cannula, where the wall of the soft cannula is pressed onto the outer surface of the rigid cannula by a compressing section of the sealing sleeve, providing a sliding sealing between the soft cannula and the rigid cannula.

A portable or wearable drug delivery device may be provided by including the cannula assembly as outlined, where the drug delivery device may be a patch pump or a patch injector or a hand-held injection device.

A method for manufacturing an improved cannula assembly may be used, where the cannula assembly comprises a soft cannula, a rigid cannula, and a sealing sleeve. The soft cannula may comprise a wall forming a soft cannula lumen configured to envelope at least a portion of the rigid cannula, and a soft cannula flange may be constructed as a portion with an extended wall thickness at or near the proximal end of the soft cannula. The sealing sleeve may be configured to envelope at least a portion of the soft cannula. The method may comprise the following steps:
bringing or inserting at least a portion of the rigid cannula into the lumen of the soft cannula
bringing or inserting at least a portion of the soft cannula into the sealing sleeve
plastically deforming at least a portion of the sealing sleeve to form a compressing section, where the wall of the soft cannula is pressed onto the outer surface of the rigid cannula providing a slidable sealing connection.

A drug delivery device with such an improved cannula assembly allows easy, damage-free and cost-effective manufacturing while ensuring optimal control of the sealing pressure and hence the reliability of drug delivery.

According to certain implementations, a cannula assembly for a portable or wearable drug delivery device may include a soft cannula; a rigid cannula; and a sealing sleeve. The soft cannula may include a wall defining a lumen configured to surround at least a portion of the rigid cannula and to provide a sliding connection. The sealing sleeve may be configured to radially surround at least a portion of the soft cannula. A flange of the soft cannula may include an extended wall thickness and is arranged at or near a proximal end of the soft cannula. An inner surface of the sealing sleeve at least partially engages an outer surface of the soft cannula and provides a scaling pressure radially between the rigid cannula and the soft cannula, thereby defining a scaling arca on an outer surface of the rigid cannula.

In various implementations and alternatives, the flange may include a proximal stop or a distal stop configured to engage a stop of the scaling sleeve, the flange may be configured to at least partially cover the sealing area, and/or the flange may include an elastic deformable material. In some cases, where the flange includes the elastic deformable material, the sealing sleeve may include a compressing section configured to receive the cannula flange in an elastically deformed or compressed state, and/or the sealing sleeve may include an entrance section configured to receive the cannula flange in an elastically undeformed or uncompressed state.

In various implementations and alternatives, the sealing sleeve may be constructed of a plastically deformable material. For instance, the plastically deformable material may be a metal. The deformable sealing sleeve may include a compressing section configured to receive the cannula flange in an elastically deformed or compressed state, and the compressing section of the sealing sleeve may be in a deformed state, and/or at least a portion of the sealing sleeve is crimped or swaged onto the soft cannula.

In various implementations and alternatives, the rigid cannula may include a 5-face cutting at a distal end to support damage-free insertion of the rigid cannula into the soft cannula.

A portable or wearable drug delivery device may include the cannula assembly described above, and the device may be configured as a patch pump or a patch injector.

Methods for manufacturing a cannula assembly for a portable or wearable drug delivery device, in some implementations, may involve the steps of: inserting at least a portion of a rigid cannula into a lumen of a soft cannula defined by a wall thereof such that the soft cannula surrounds at least the portion of the rigid cannula. The soft cannula may include a flange having an extended wall thickness at or near a proximal end of the soft cannula; inserting at least a portion of the soft cannula into a sealing sleeve such that the sealing surrounds at least the portion of the soft cannula; and plastically deforming at least a portion of the sealing sleeve to form a compressing section. The wall of the soft cannula may be pressed onto an outer surface of the rigid cannula thereby providing a slidable sealing connection.

In some cases, the flange may include a distal or a proximal stop configured to engage the scaling sleeve, the flange may be configured to at least partially cover a sealing area on an outer surface of the rigid cannula, and/or the flange may be constructed of an elastic deformable material.

### BRIEF DESCRIPTION OF THE DRAWINGS

The subject matter of the invention will be explained in more detail in the following text with reference to preferred exemplary embodiments which are illustrated in the attached drawings, in which:

FIGS. 1a and 1b depict a modular patch pump according to the present invention;

FIG. 1a depicts a three-dimensional outside view of the patch pump; and

FIG. 1b depicts a sectional view of the patch pump.

FIG. 2 depicts the pump unit and the reservoir unit of the patch pump.

FIG. 3 depicts a view of the pump unit with the housing removed.

FIGS. 4a and 4b depict the reservoir unit with selected parts removed for illustration;

FIG. 4a depicts the reservoir unit with the housing removed; and

FIG. 4b depicts an arrangement of the cannula assembly inside the reservoir unit.

FIGS. 5a and 5b depict a first embodiment of the cannula assembly;

FIG. 5a depicts the first cannula assembly with a sealing sleeve before deformation; and

FIG. 5b depicts the first cannula assembly with a sealing sleeve after deformation.

FIGS. 6a and 6b depict a second embodiment of the cannula assembly;

FIG. 6a depicts the second cannula assembly with a sealing sleeve before deformation; and

FIG. 6b depicts the second cannula assembly with a sealing sleeve after deformation.

FIGS. 7a and 7b depict a third embodiment of the cannula assembly;

FIG. 7a depicts the third cannula assembly with a sealing sleeve before axial insertion of the sealing flange; and

FIG. 7b depicts the third cannula assembly with a sealing sleeve after axial insertion of the sealing flange.

FIGS. 8a and 8b depict examples of shapes for crimping the sealing sleeve;

FIG. 8a depicts a sealing sleeve in a “tulip” shape after incomplete crimping; and

FIG. 8b depicts a sealing sleeve after crimping over the complete length.

The reference symbols used in the drawings, and their primary meanings, are listed in summary form in the list of designations. In principle, identical parts are provided with the same reference symbols in the figures.

### DETAILED DESCRIPTION

### Definitions

In the present context, the terms “substance”, “drug”, “medicament” and “medication” are to be understood to include any flowable medical formulation suitable for controlled administration through a means such as, for example, a cannula or a hollow needle, and comprises a liquid, a solution, a gel or a fine suspension containing one or more medical active ingredients. A medicament can be a composition comprising a single active ingredient or a pre-mixed or co-formulated composition with more than one active ingredient present in a single container. Medication includes drugs such as peptides (e.g., insulin, insulin-containing drugs, GLP-1 containing drugs or derived or analogous preparations), proteins and hormones, active ingredients derived from, or harvested by, biological sources, active ingredients based on hormones or genes, nutritional formulations, enzymes and other substances in both solid (suspended) or liquid form but also polysaccharides, vaccines, DNA, RNA, oligonucleotides, antibodies or parts of antibodies but also appropriate basic, auxiliary and carrier substances.

The term “distal” is meant to refer to the direction or the end of the drug delivery device carrying an injection needle or an injection cannula, whereas the term “proximal” is meant to refer to the opposite direction or end pointing away from the needle or cannula.

The term “injection system” or “injector” refers to a device that is removed from the injection site after each medication event or drug delivery process, whereas the term “infusion system” refers to a device with a cannula or needle that remains in the skin of the patient for a prolonged period of time, for example, several hours.

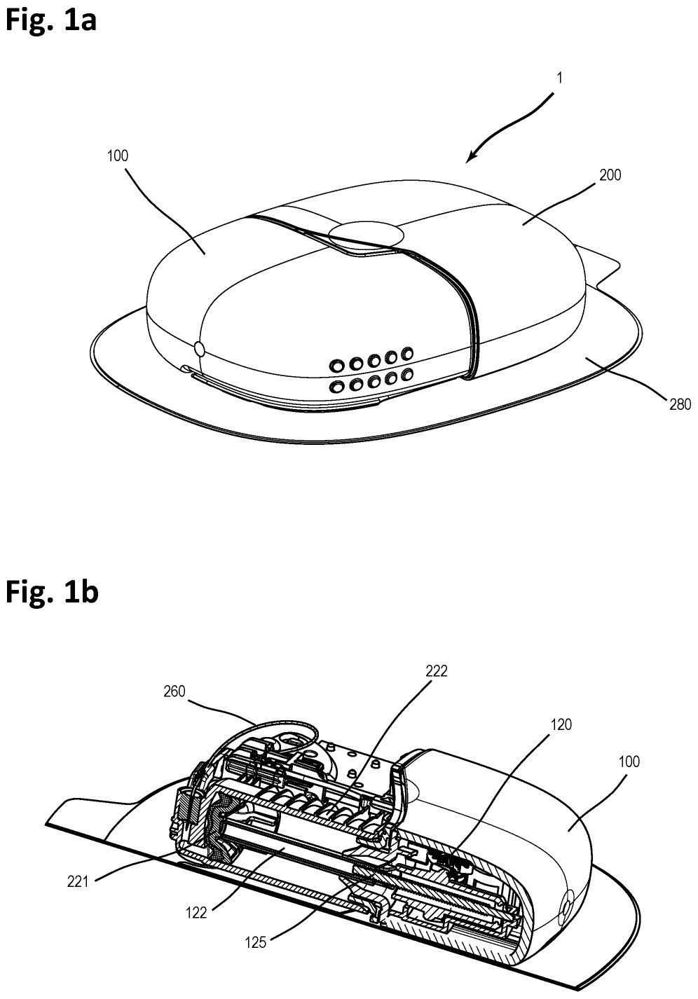

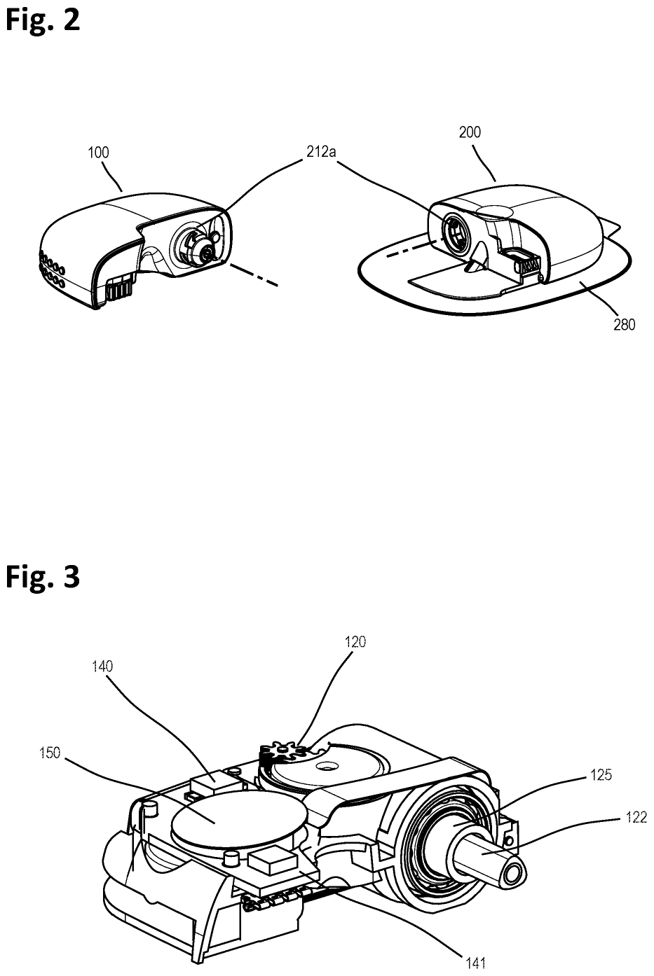

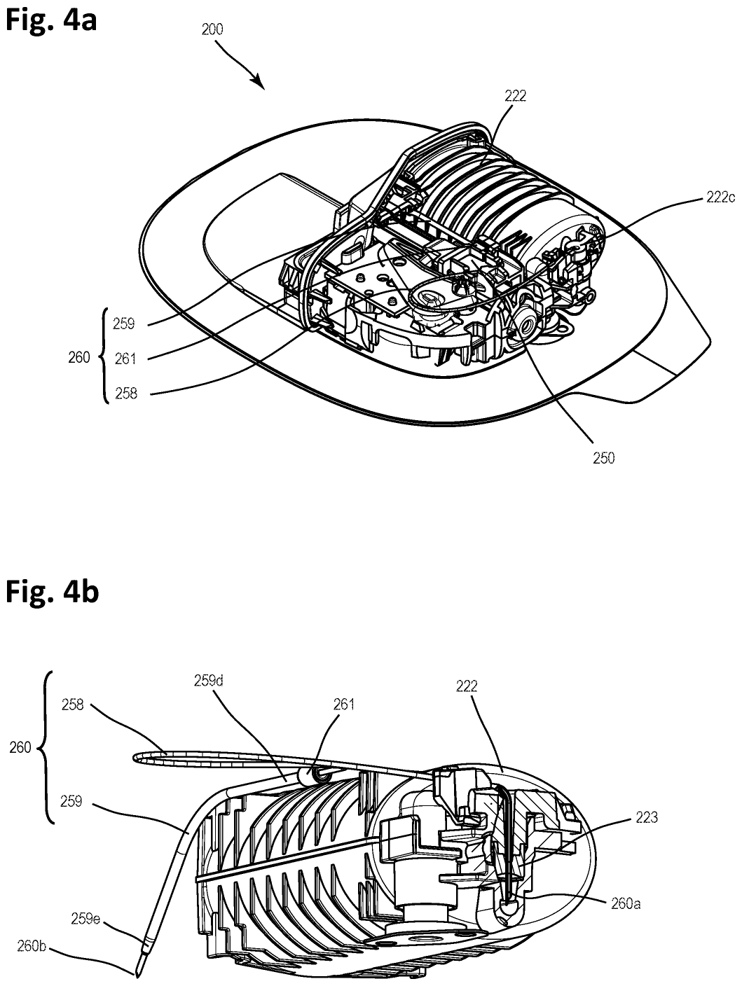

Turning to the figures, FIG. 1a depicts a drug delivery device including a cannula assembly according to the present disclosure. The drug delivery device is configured as a patch pump (1) attachable to the skin of a patient. The patch pump (1) may include a reusable pump unit (100) releasably connected via a bayonet connection (212a, see FIG. 2) to a disposable reservoir unit (200). The reservoir unit (200) may include a reservoir to store the medicament and a needle assembly with a fluid path to bring the drug from the reservoir into the body of the patient. At the bottom of the reservoir unit (200) an adhesive patch assembly (280) is included to attach the patch pump (1) to the body of the patient. The pump unit (100) may be releasably and sealingly connected to the reservoir unit (200) by a bayonet connection. FIG. 1a shows the complete patch pump (1) with both units joined together, seen from a position above the pump. In the context of the present invention, “above” or “top” refers to the side of the pump which is facing away from the patient's body when the pump is attached to the patient for drug delivery. Consequently, “bottom” or “base” refers to the side of the pump facing towards the patient's body during drug delivery. FIG. 1b depicts a cut through the patch pump (1) to show the arrangement of main inner parts of the drug delivery device of FIG. 1a. With reference to FIG. 3, the pump unit (100) may include a drive mechanism (120) for driving the plunger rod (122), an encoder to monitor the movement of the drive mechanism, a rechargeable battery and a control unit configured to control the set-up, drug delivery and monitoring of the pump. The battery may be rechargeable and may be configured to be charged by a further battery in the disposable reservoir unit (200) while the drive mechanism (120) is connected to the reservoir unit (200). The drive mechanism (120) is actuated by an electric motor and acts mechanically by transforming the rotation of the motor via a gearbox assembly and a threaded rod (125) to a linear displacement of a plunger rod (122) and from there to a plunger in the reservoir (222) to dispense the medical substance out of the reservoir (222). A gearbox assembly is particularly useful to provide for small displacements corresponding to small amounts of drug. The plunger rod may include just one threaded rod (125), but alternative embodiments may include a plunger rod assembly including multiple segments. While any electric motor may be used to drive the drug delivery device, brushless DC or stepper motors are usually preferred for safety reasons. A needle assembly (260) inside the reservoir unit (200) provides the fluid connection from the reservoir (222) to the exterior of the pump for application to the patient. To ensure safe handling, the patch pump (1) is manufactured, shipped, stored and prepared for use with the needle assembly (260) completely inside the enveloping shape of the pump (1). The enveloping shape is an imaginary surface enveloping the housing of the pump (1) while smoothly bridging all gaps and recesses, should any be present, to a closed shell. It is the shape of the pump (1) as perceived by the user from a distance and relevant when it comes to aspects of use like handling or wearability. Preparation of the patch pump in this embodiment includes filling the reservoir inside the disposable reservoir unit (200) from the exterior using a transfer syringe and attaching the pump to the body of the patient by means of the adhesive patch assembly (280). An insertion assembly (250, see FIG. 4a) is included in the disposable reservoir unit (200) and configured to bring an output portion (260b) of the needle assembly (260), in particular the open distal end of the needle assembly (260), out of the enveloping shape of the pump and into the body of the patient once the pump is ready to start drug delivery. In a preferred embodiment of the patch pump (1) the needle assembly (260) includes a rigid cannula (258) and a soft cannula (259), and the insertion assembly (250) is configured to insert the distal end of the soft cannula into the body of the patient using the rigid cannula, which will subsequently be retracted for drug delivery.

FIG. 2 illustrates the semi-disposable patch pump from FIG. 1 with the pump unit (100) and the reservoir unit (200) detached and separated, in a view from above, with the adhesive patch assembly (280) at the bottom towards the body of the patient, and the bayonet connection (212a) in a disengaged state.

FIG. 3 further illustrates the pump unit of FIG. 1, with selected parts removed to show the inner components. In particular, the drive mechanism (120) is shown with the driving means configured as a threaded rod (125) mounted in, and in cooperation with a plunger rod (122), with the rechargeable battery (150) and the system control circuitry (140) on a printed circuit board (141). The threaded rod (125) and the plunger rod (122) are arranged substantially parallel to the rotation axis of the bayonet connection.

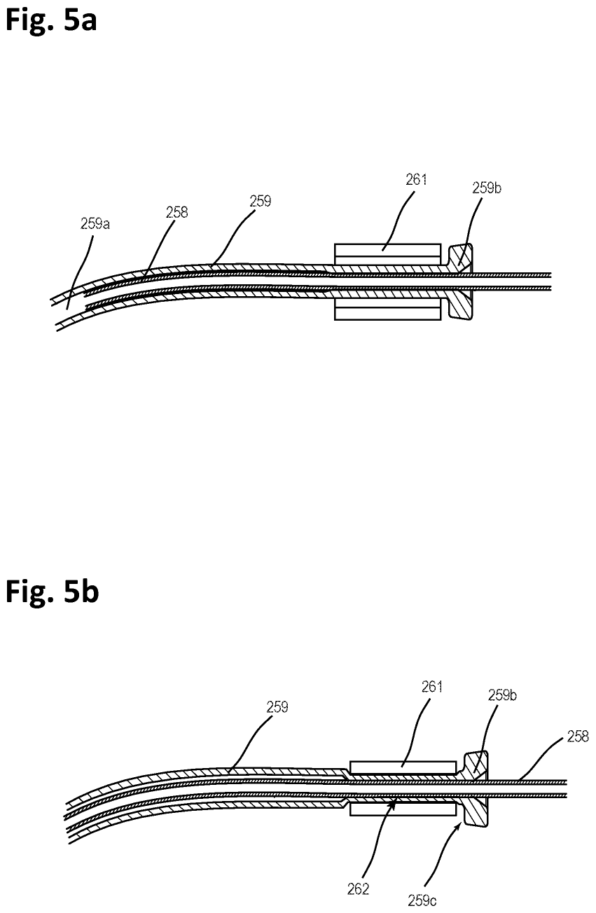

Turning to the reservoir unit (200), FIG. 4a gives an overview providing a perspective view with the housing of the reservoir unit removed, in a state before use, when the complete needle assembly (260) is inside the reservoir unit housing, with the proximal end of the rigid cannula (258) connected to the reservoir outlet (222c) and with the soft cannula (259) ready for insertion into the body of a patient. FIG. 4a shows the inner parts of the reservoir unit from outside, FIG. 4b show a similar view of the reservoir unit with a sectional cut across the outlet of the reservoir (222) to show the configuration of the rigid cannula (258), with the proximal end of the rigid cannula forming the input portion (260a) of the needle assembly (260). The reservoir outlet sealing (223) ensures a fluid-tight connection between the reservoir (222) and the needle assembly (260). The needle assembly (260) is shown in a retracted position before insertion into the body of a patient. In this position, the entire soft cannula is slidably mounted on the rigid cannula. The tip (260b) of the rigid cannula (260) cannula protrudes at the distal end of the needle assembly, ready for piercing the soft tissue of the patient. The proximal end of the soft cannula (259) is arranged towards the proximal end of the rigid cannula (258). Both the soft cannula (259) and the rigid cannula (258) are operatively connected to the insertion mechanism or insertion assembly (250), which controls the movement of the needle assembly (260) during insertion and retraction, and keeps the components of the needle assembly in a well-defined position before, during and after drug delivery. The improved needle assembly (260) includes three main components: a soft cannula (259), a rigid cannula (258) and a sealing sleeve (261). During insertion, all three components are moved forward in a distal direction. With the soft cannula (259) successfully inserted into the body of a patient, the rigid cannula (260) is retracted. FIGS. 5a-5b, FIGS. 6a-6b and FIGS. 7a-7b show three different embodiments of such a needle assembly (260). The soft cannula (259) is made of an at least partially elastic material, such as a thermoplastic Fluorinated Ethylene Propylene (FEP) or Polypropylene (PP), and substantially shaped as a tube with a wall surrounding a lumen inside. The rigid cannula is substantially a hollow needle made of a rigid material, such as stainless steel or a rigid plastic. The sliding connection between the rigid cannula (258) and the soft cannula (259) is provided by making the lumen of the soft cannula large enough to allow insertion of the rigid cannula. To achieve a fluid-tight sealing between the two components, a sealing pressure is required, pressing the wall of the soft cannula (259) onto the surface of the rigid cannula (258). These two requirements-easy insertion and fluid-tight sealing—are conflicting and difficult to fulfil in one single mechanical design. The cannula assembly (260) may therefore be improved by introducing a scaling sleeve (261), configured to envelope at least a part of the soft cannula. For manufacturing, the sealing sleeve (261) is held in an open state or in a state where the sealing sleeve does not apply a pressure onto the soft cannula. The inner dimension, for example inner diameter of the sealing sleeve is large enough to allow easy insertion of the combined soft and rigid cannula. For use, the sealing sleeve (261) is brought into a position or state where at least a section of the sealing sleeve (261) is radially pressing onto the soft cannula, thereby providing a scaling pressure and establishing a slidable scaling between the rigid cannula (258) and the soft cannula (259). The area on the surface of the rigid cannula where the wall of the soft cannula (259) is pressed onto the rigid cannula shall be termed “scaling area” (262). FIGS. 5a and 5b illustrate a first embodiment of such a cannula assembly, where the sealing sleeve (261) is made of a plastic deformable material, such as aluminium or stainless steel. In an initial state, shown in FIG. 5a, the sealing sleeve (261) is provided with an inner diameter large enough to allow the soft cannula and the rigid cannula to be inserted without applying any pressure. In a second step, the sealing sleeve (261) is plastically deformed, for example crimped or swaged onto the soft cannula, which results in a radial sealing pressure from the sealing sleeve via the soft cannula to the outer surface of the rigid cannula. This state is shown in FIG. 5b. In this example, the sealing area (262) is next to the soft cannula flange (259b), axially separated, showing that these two features do not need to overlap. A distal stop (259c) may ensure that the soft cannula (259) does not slide backwards when the cannula assembly is pushed in a distal direction for insertion into the tissue of a patient. In this state, the cannula assembly provides a sliding but at the same time reliably fluid-tight sealing, ready for safe and reliable drug delivery and related functions such as occlusion detection, priming of the device or filling of the reservoir.

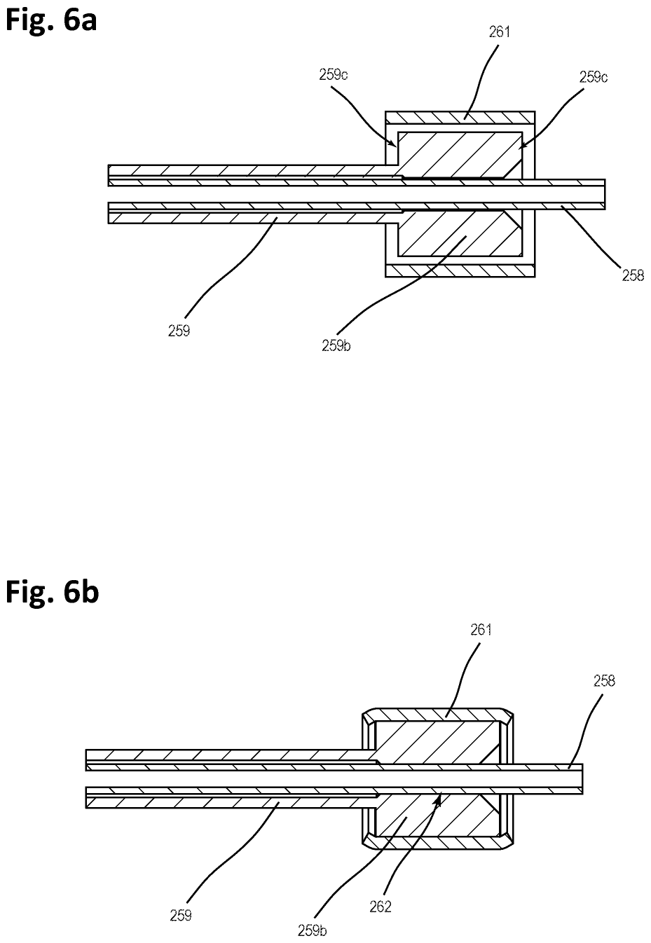

In the embodiment of FIGS. 5a and 5b, deformation of the sealing sleeve (261) may need to be rather uniform over its circumference because the wall of the soft cannula (259) over the sealing area (262) is generally thin. Moving the sealing sleeve (261) over the soft cannula flange (259b) provides much more wall thickness that is available for uniform compression in the sealing area (262) and allows for more variation or dimensional tolerance of the inner diameter of the sealing sleeve even in a state for use. The elastic properties of the soft cannula flange (259b) may compensate these dimensional variations and ensure a proper sealing pressure all around the rigid cannula. This is illustrated in FIGS. 6a and 6b, with FIG. 6a showing such an embodiment in a manufacturing state before deformation of the sealing sleeve, and FIG. 6b showing the same embodiment after deformation of the sealing sleeve (261). With the sealing sleeve (261) overlapping the soft cannula flange (259b), both axial ends of the soft cannula flange (259b) may have a proximal or distal stop (259c, FIG. 6a) to engage with the deformed ends of the sealing sleeve (261, FIG. 6b). This facilitates the fixation of the soft cannula (259) to the insertion assembly (250) while still providing a reliable slidable connection between the soft cannula (259) and the rigid cannula (258). Just like in the example of FIG. 5b, the cannula assembly of FIG. 6b provides a sliding but at the same time reliably fluid-tight sealing.

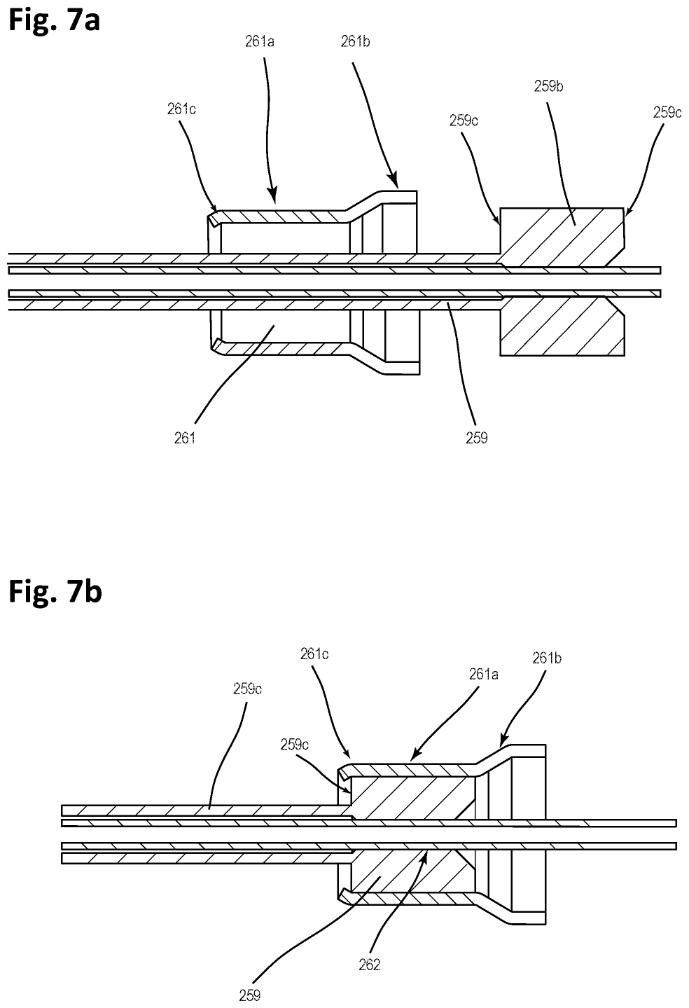

FIG. 7 illustrates that the concept of combining a soft cannula flange with a sealing sleeve is not restricted to examples where the sealing sleeve is deformed during manufacturing. In this example, the sealing sleeve (261) has a substantially cylindrical shape with two diameters and a gradual decrease from a larger diameter in an entrance section (261b) to a compressing section (261a). At the distal end of the sealing sleeve (261), a second reduction of diameter, a sleeve stop (261c) or an end wall may be provided. At the proximal end of an entrance section (261b), the inner diameter is configured large enough to receive the soft cannula and the rigid cannula for damage-free insertion, typically a 0.1 to 1 mm larger than the outer diameter of the soft cannula flange in a relaxed, uncompressed state. During manufacturing, the rigid cannula is first inserted into the lumen of the soft cannula. The two cannulas are then axially inserted together into the inner volume of the scaling sleeve. This is the state shown in FIG. 7a. The insertion will typically be continued by pushing the soft cannula at the proximal stop (259c) of the soft cannula (259) in the distal direction into the open entrance section (261b) of the sealing flange (261), and further into the compression section (261a) until a sufficient length of the soft cannula flange (259b) is compressed, typically until the distal stop (259c) of the soft cannula flange (259) reaches the sleeve stop (261c). This is the state shown in FIG. 7b. Pressing the soft cannula flange (259) into the compressing section (261a) of the sealing sleeve (261) provides a radial sealing pressure with thereby forming sealing area (262) without deforming the sealing sleeve (261). As the compressing section (261a) may itself be of a cylindrical shape, compressing pressure will typically be substantially uniform over the complete circumference of the soft cannula flange, which may further improve the fluid-tight sealing between the soft cannula (259) and the rigid cannula (258). The proximal and/or distal stop (259c) improves fixation of the soft cannula (259) to the insertion assembly (250) and ensures a sliding but at the same time reliably fluid-tight sealing. The order of manufacturing steps as described may be chosen differently, and combinations of steps may be chosen-such as pre-crimping a scaling sleeve to a shape according to FIG. 7a before inserting the cannulas, or applying a crimping step after completing assembly as shown in FIG. 7b. Many other examples are possible by adding a soft cannula flange to a soft cannula, and by adding a scaling sleeve to provide a sealing pressure, without leaving the scope of the invention as claimed.

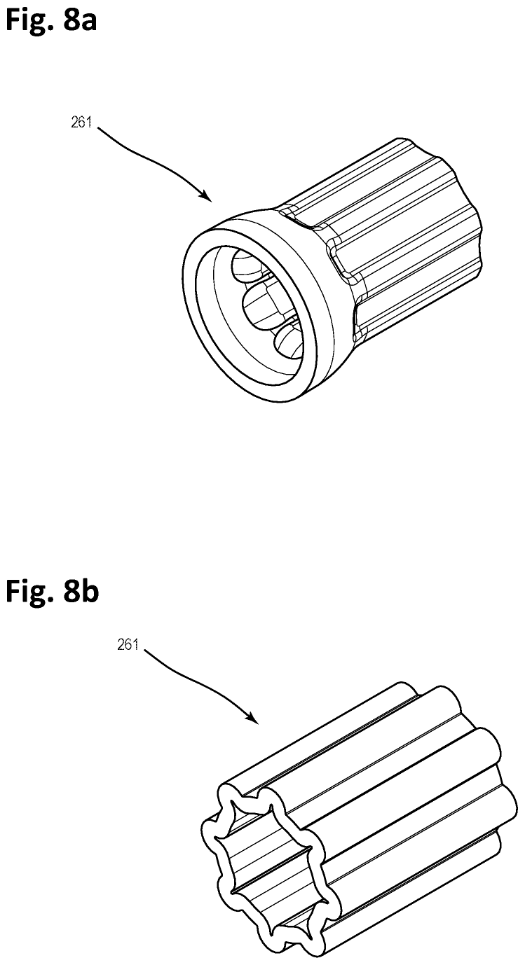

Another way to implement variations of the implementations of the present disclosure is to vary the form of deformation of the sealing sleeve (261). A process called swaging may be applied instead of crimping, resulting in a more uniform deformation similar to the shape shown in FIGS. 7a and 7b, with the same improving effect on uniformity of scaling pressure. When it comes to crimping the scaling sleeve, any deformation is possible which reduces the inner dimensions of the sealing sleeve and thereby creates a sealing pressure. It is a direct effect of the increased wall thickness of the soft cannula flange (259b) with the reduced inner dimension of the scaling sleeve (262) that the shape of deformation is less of an issue, because the elasticity of the soft cannula flange will help ensuring that a minimal pressure is provided over the sealing area completely covering the circumference of the rigid cannula. Further, crimping may not be applied over the complete length of the sealing sleeve, but only on a specific compressing section. FIGS. 8a and 8b illustrate two possible shapes for crimping the sealing sleeve: crimping an axial section of the sealing sleeve may result in a “tulip” shape, as shown in FIG. 8a, and a complete crimping over the full length of the sealing sleeve in FIG. 8b. Crimping a section of the sealing sleeve may improve the axial fixation of the soft cannula with respect to the insertion assembly (250), just like the proximal or distal stop of the soft cannula flange described before. Further manufacturing steps may be included such as measuring or monitoring the tightness of the seal between the soft cannula and the rigid cannula during crimping such that each sealing sleeve is individually and axially crimped up to the level for reaching a uniform tightness of the seal.

While the invention has been described in detail in the drawings and foregoing description, such description is to be considered illustrative or exemplary and not restrictive. Variations to the disclosed embodiments can be understood and effected by those skilled in the art and practising the claimed invention, from a study of the drawings, the disclosure, and the appended claims. In the claims, the word “comprising” does not exclude other elements or steps, and the indefinite article “a” or “an” does not exclude a plurality. The mere fact that certain elements or steps are recited in distinct claims does not indicate that a combination of these elements or steps cannot be used to advantage, specifically, in addition to the actual claim dependency, any further meaningful claim combination shall be considered disclosed.

### LIST OF REFERENCE NUMERALS

1 Patch pump
100 Pump unit
120 Drive mechanism
122 Plunger rod
125 Threaded rod
140 System control circuitry
141 Printed circuit board, PCB-PU
150 Rechargeable battery
200 Reservoir unit
212a Bayonet connection
221 Plunger
222 Reservoir
222c Reservoir outlet
223 Reservoir outlet sealing
250 Insertion assembly
258 Rigid cannula
259 Soft cannula
259a Soft cannula lumen
259b Soft cannula flange
259c Proximal or distal stop
259d proximal end of soft cannula
259e distal end of soft cannula, output portion of the needle assembly
260 Cannula assembly or needle assembly
260a Input portion
260b Output portion
261 Sealing sleeve
261a compressing section
261b entrance section
261c sleeve stop
262 Scaling area
280 Adhesive patch assembly

1. A cannula assembly for a portable or wearable drug delivery device, comprising:
a soft cannula;
a rigid cannula; and
a sealing sleeve,
wherein the soft cannula comprises a wall defining a lumen configured to surround at least a portion of the rigid cannula and to provide a sliding connection,
wherein the sealing sleeve is configured to radially surround at least a portion of the soft cannula,
wherein a flange of the soft cannula comprises an extended wall thickness and is arranged at or near a proximal end of the soft cannula, and
wherein an inner surface of the sealing sleeve at least partially engages an outer surface of the soft cannula and provides a sealing pressure radially between the rigid cannula and the soft cannula, thereby defining a sealing area on an outer surface of the rigid cannula.

2. The cannula assembly according to claim 1, wherein the flange comprises a proximal stop or a distal stop configured to engage a stop of the sealing sleeve.

3. The cannula assembly according to claim 1, wherein the flange is configured to at least partially cover the sealing area.

4. The cannula assembly according to claim 1, wherein the flange comprises an elastic deformable material.

5. The cannula assembly according to claim 4, wherein the sealing sleeve comprises a compressing section configured to receive the cannula flange in an elastically deformed or compressed state.

6. The cannula assembly according to claim 4, wherein the sealing sleeve comprises an entrance section configured to receive the cannula flange in an elastically undeformed or uncompressed state.

7. The cannula assembly according to claim 1, wherein the sealing sleeve is constructed of a plastically deformable material.

8. The cannula assembly according to claim 7, wherein the plastically deformable material is a metal.

9. The cannula assembly according to claim 7, wherein the sealing sleeve comprises a compressing section configured to receive the cannula flange in an elastically deformed or compressed state, and wherein the compressing section of the sealing sleeve is in a deformed state.

10. The cannula assembly according to claim 7, wherein at least a portion of the sealing sleeve is crimped or swaged onto the soft cannula.

11. The cannula assembly according to claim 1, wherein the rigid cannula comprises a 5-face cutting at a distal end to support damage-free insertion of the rigid cannula into the soft cannula.

12. A portable or wearable drug delivery device comprising the cannula assembly of claim 1, wherein the device is configured as a patch pump or a patch injector.

13. A method for manufacturing a cannula assembly for a portable or wearable drug delivery device, the method comprising the steps of:
inserting at least a portion of a rigid cannula into a lumen of a soft cannula defined by a wall thereof such that the soft cannula surrounds at least the portion of the rigid cannula, wherein the soft cannula comprises a flange having an extended wall thickness at or near a proximal end of the soft cannula;
inserting at least a portion of the soft cannula into a sealing sleeve such that the sealing surrounds at least the portion of the soft cannula; and
plastically deforming at least a portion of the sealing sleeve to form a compressing section, wherein the wall of the soft cannula is pressed onto an outer surface of the rigid cannula thereby providing a slidable sealing connection.

14. The method according to claim 13, wherein the flange comprises a distal or a proximal stop configured to engage the scaling sleeve.

15. The method according to claim 13, wherein the flange is configured to at least partially cover a sealing area on an outer surface of the rigid cannula.

16. The method according to claim 13, wherein the flange is constructed of an elastic deformable material.
